SEQUENCE LISTING (4 sequences)

SEQ ID NO: 1 - protein, 12 aa, Artificial Sequence
  MOD_RES 1: Xaa =“Ac-Arg”. AC is acetylated.
XIARGDFPDDRX

SEQ ID NO: 2 - protein, 5 aa, Artificial Sequence
  MOD_RES 1: Xaa = an amino acid or a derivatized or chemically modified amino acid
XRGDX

SEQ ID NO: 3 - protein, 12 aa, Artificial Sequence
  MOD_RES 1: Xaa = “TFA-Arg” and “Ac-Arg” note TFA is trifluoroacetic acid.
XIAXGDFPDDXX

SEQ ID NO: 4 - protein, 12 aa, Artificial Sequence
  MOD_RES 1: Xaa = “TFA-Arg” and “Ac-Arg” /note TFA is trifluoracetic acid. Ac is acylated.
XIAXGDFPDDXX

(19) United States
(12) Patent Application Publication
(10) Pub. No.: US 2003/0170250 A1
(43) Pub. Date: Sep. 11, 2003

(54) Local delivery of long lasting therapeutic agents

(76) Inventors: Alan M. Ezrin, Moraga, CA (US); Peter G. Milner, Los Altos Hills, CA (US); Dominique P. Bridon, Outremont (CA); Darren L. Holmes, Montreal (CA)

Correspondence Address: Michael R. Ward, Morrison & Foerster LLP, 425 Market Street, San Francisco, CA, 94105-2482, US

(21) Appl. No.: 10/152,232

(22) Filed: May 20, 2002

Related U.S. Application Data
(63) Continuation of application No. 09/424,571, filed on Mar. 7, 2000, now abandoned.
Application No. 09/424,571 is a 371 of international application No. PCT/US99/06344, filed on Mar. 23, 1999.
(60) Provisional application No. 60/078,974, filed on Mar. 23, 1998.
(60) Provisional application No. 60/086,352, filed on May 20, 1998.
(60) Provisional application No. 60/086,205, filed on May 21, 1998.
(60) Provisional application No. 60/107,391, filed on Nov. 6, 1998.

(51) Int. Cl. A61K 39/00

(52) U.S. Cl. 424/184.1

(57) ABSTRACT

Methods of and compositions for localized delivery of therapeutic agents which are capable of forming covalent bonds with a site of interest are disclosed. Therapeutic agents useful in the invention include wound healing agents, antibiotics, anti-inflammatories, anti-oxidants, anti-proliferatives, immunosupressants, anti-infective and anti-cancer agents.

### FIELD OF THE INVENTION

This invention relates to the field of therapeutic agents in medicine. In particular, this invention relates to the field of localized delivery of therapeutic agents wherein the agents are capable of covalently bonding to a site of interest in vivo, to provide increased tissue retention and pharmacodynamic duration of therapeutic benefit for the given drug.

### BACKGROUND OF THE INVENTION

The technology of local delivery of a therapeutic using drug delivery catheters or devices is well established. Under ideal circumstances, the therapeutic agent will remain near the site of administration for increased effectiveness. While useful, the main drawback of this technology is the rate at which the therapeutic agent is washed away from the site of application. For example, according to Imanishi et al. (J Cardio, 1996, 27, 267-271), the concentration of residual argatroban introduced through pressure balloon catheter is decreased by three folds in the first five minutes after deflation of the balloon. This phenomenon is reported repeatedly in the literature for other therapeutic agents resulting in a general deficiency that drugs from a diverse therapeutic areas have limited utility following localized delivery due to their inability to maintain adequate concentrations within hours after delivery (Circulation, 1994, 89 (4), 1518-1524; Circulation, 1997, 96 (1), 154-165). As a result, repeated dosings of locally administered therapeutic agents are required in order to avoid rapid reduction of drug levels and sub-optimal performance or sub-effective responsiveness. This results in increased costs and unnecessary patient exposure to excessive amounts of therapeutic agents.

Thus, there is a need to provide therapeutic agents to localized sites such that the therapeutic agents have increased retention at the desired site and prolonged duration of action. These therapeutic agents are not as easily washed away from the site of administration so that reduced amounts of the agents can be supplied. In particular, there is a need to provide therapeutic agents capable of forming covalent bonds to localized sites such that the therapeutic agents have increased effective presence for therapeutic benefits.

### SUMMARY OF THE INVENTION

In order to meet these needs, the present invention is directed to therapeutic agents capable of forming covalent bonds to localized sites such that the therapeutic agents have increased tissue retention and half-lifes.

A first aspect of this invention relates to the modification of drugs at a site not involved in the pharmacophore receptor interaction.

A second aspect of this invention relates to novel chemistry involved in the non-specific formation of covalent bonds using homo and heterobifunctional cross-linking reagents.

In addition, the invention includes the non-specific labeling of fixed blood proteins and tissues with N-Hydroxy-Succinimide (NHS)-drugs and sulfo-NHS drugs, although other reactive groups, which are functional in an aqueous medium such as blood, may also be employed. In some cases, special reagents find use, such as azido, diazo, carbodiimide anhydride, hydrazine, dialdehydes, thiol groups, or amines to form amides, esters, imines, thioethers, disulfides, substituted amines, or the like. Usually, the covalent bond which is formed should be able to be maintained during the lifetime of the blood component, unless it is intended to be a release site. A major advantage of this technology is the small amount of drug necessary to provide an effective therapeutic window when compared to systemic administration. The reasons for this advantage are explained by the targeting of the delivery, the high yield of reaction between reactive entity and reactive functionality and the irreversible nature of the bond formed after reaction. The therapeutic agent is localized in the vicinity of the therapeutic target and is not allowed to circulate in the blood stream like any other free drug. Another advantage of the technology is its limited side effects that is a consequence of the advantage described above. Once bound to the membrane or tissue the therapeutic agent is not susceptible to liver metabolism, kidney filtration and excretion, and may even be protected from protease (inclusive of endopeptidase) activity which usually leads to loss of activity and accelerated elimination.

Another aspect of this invention relates to novel chemically reactive derivatives of radiolabeled molecules which can react with available functionalities on fixed blood proteins and on tissues to form covalent linkages, and in which the resulting covalently bound conjugates have radioactive properties.

As compared with free radiolabeled drugs the conjugated molecules have extended lifetimes in the bloodstream and are, therefore, capable of maintaining the radioactivity for extended periods of time as compared to the unconjugated parent drug, and provide such activity with reduced centrally mediated side effects.

In addition, the invention includes the non-specific labeling of fixed blood proteins and tissues with NHS-radiolabeled drugs and sulfo-NHS radiolabeled drugs although other reactive groups, which are functional in an aqueous medium such as blood, may also be employed. In some cases, special reagents find use, such as azido, diazo, carbodiimide, anhydride, hydrazine, dialdehydes, thiol groups, or amines to form amides, esters, imines, thioethers, disulfides, substituted amines, or the like. Usually, the covalent bond which is formed should be able to be maintained during the lifetime of the blood or tissue component, unless it is intended to be a release site.

The invention further includes the conjugates of the radiolabeled derivatives with fixed blood components and tissues and methods for providing radioactivity in vivo comprising administering to a mammalian host the novel radiolabeled derivatives through the use of percutaneous catheter technology, endarterectomy or direct tissue incubation.

Another aspect of this invention relates to novel chemically reactive derivatives of RGD-containing peptides which can react with available functionalities on fixed blood proteins and on tissues to form covalent linkages, and in which the resulting covalently bound conjugates have RGD peptide activity.

As compared with RGD peptide drugs the conjugated molecules have extended lifetimes in the bloodstream or tissues and are, therefore, capable of maintaining RGD peptide activity for extended periods of time as compared to the unconjugated parent drug. Such activity is provided with reduced centrally mediated side effects due to the high efficiency of localized delivery, the irreversible covalent bond between therapeutic agent and membrane protein and the lower therapeutic doses used with this invention.

In addition, the invention includes the non-specific labeling of fixed blood proteins and tissues with NHS-RGD peptides and sulfo-NHS peptides although other reactive groups, which are functional in an aqueous medium such as blood, may also be employed. In some cases, special reagents find use, such as azido, diazo, carbodiimide anhydride, hydrazine, dialdehydes, thiol groups, or amines to form amides, esters, imines, thioethers, disulfides, substituted amines, or the like. Usually, the covalent bond which is formed should be able to be maintained during the lifetime of the blood or tissue component, unless it is intended to be a release site.

The invention further includes the conjugates of the RGD peptide derivatives with fixed blood components and tissues and methods for providing RGD peptide activity in vivo comprising administering to a mammalian host the novel RGD peptide derivatives through the use of percutaneous catheter technology, endarterectomy, pericardial, advantitial, intracardiac or direct tissue incubation.

The invention further includes the method of use for conjugates of RGD derivatives with fixed blood components and tissues for providing RGD peptide to perform wound healing through platelet immobilization generated by the binding of the peptide onto glycoprotein IIb/IIIa receptors.

The invention further includes the method of use for conjugates of RGD derivatives with fixed blood components and tissues for providing RGD peptide to produce anti-restenosis properties through direct interaction with known integrin receptors (alpha V beta III, IIb/IIIa, alpha 5 beta III etc.) to modulate the stimulation of platelets and inflammatory responses including induction of proliferation, migration, cell adhesion and tissue remodeling.

The invention further includes the method of use for conjugates of RGD derivatives with fixed blood components and tissues for providing RGD peptide to produce anti-angiogenic and antimetastatic properties through modification of adhesion and migration mediated by integrin receptor interaction with cell adhesion and cell to cell coupling as well as matrix degradation associated with cellular immigration and emigration.

The invention further includes a local delivery agent comprising a compound of the formula: X—Y-Z wherein X is selected from the group of wound healing agents, antibiotics, anti-inflammatories, antioxidants, anti-proliferatives, anti-restenosis, antiangiogenic, immunosuppressants, anti-infectives and anti-cancer agents. In the formula, Y is a linking group consisting of 0-30 atoms and Z is a chemically reactive entity capable of reaction with a reactive functionality on fixed blood components to form covalent bonds therewith.

In one format, Z may be selected from N-hydroxysuccinimide, N-hydroxy sulfosuccinimide, maleimide-benzoyl-succinimide, gamma-maleimido-butyryloxy succinimide ester, maleimidopropionic acid, isocyanate, thiolester, thionocarboxylic acid ester, imino ester, carbodiimide anhydride and carbonate ester.

In another format, X may be selected from peptides, organic molecules and radioactive molecules. In another format, X may be an RGD containing peptide having wound healing properties. The RGD peptide may have the sequence Ac-RIARGDFPDDRK(EGS)-NH₂ where EGS is ethylene glycol-bis(succinimidylsuccinate).

The invention further includes a method of increasing the retention time of a therapeutic agent locally administered to a site by delivering a compound of the formula: X—Y-Z wherein X is selected from the group of wound healing agents, antibiotics, anti-inflammatories, antioxidants, anti-proliferatives, anti-restenosis, antiangiogenic, immunosuppressants, anti-infectives and anti-cancer agents. In the formula, Y is a linking group consisting of 0-30 atoms and Z is a chemically reactive entity capable of reaction with a reactive functionality on fixed blood components to form covalent bonds therewith.

The invention further includes a method of promoting wound healing at a wound site, comprising administering a compound of the formula: X—Y-Z wherein X is a wound healing agent, Y is a linking group of 0-30 atoms and Z is a chemically reactive entity capable of reaction with a reactive functionality on fixed blood components to form covalent bonds therewith.

The invention further includes a method of treating a tumor comprising a compound of the formula X—Y-Z wherein X is an anticancer agent, Y is a linking group of 0-30 atoms and Z is a chemically reactive entity capable of reaction with a reactive functionality on fixed blood components to form covalent bonds therewith.

### BRIEF DESCRIPTION OF THE DRAWINGS

The invention will be better understood by reference to the figures, in which:

FIG. 1 represents the association efficiency of [³H]-NHS-propionate with the damaged rabbit carotid arteries following local incubation.

FIG. 2 represents the retention efficiency of [³H]-NHS-propionate following a 3 minutes incubation period in damaged rabbit carotid arteries.

FIG. 3 represents the 3 days retention efficiency of [³H]-NHS-propionate and [3H]-propionate following a 3 minutes incubation period with damaged rabbit carotid arteries.

### DETAILED DESCRIPTION OF THE INVENTION

Definitions

To ensure a complete understanding of the invention the following definitions are provided:

Local Delivery Agent: Local delivery agents are agents that may be delivered to a local site of interest. Such agents include therapeutic agents. Local delivery includes topical application to both internal and external sites requiring therapeutic treatment.

Therapeutic Agents: Therapeutic agents are agents that have a therapeutic effect. Therapeutic agents include wound healing agents, antibiotics, anti-infectives, anti-oxidants, chemotherapeutic agents, anti-cancer agents, anti-inflammatory agents, and antiproliferative drugs.

Fixed blood components: Fixed blood components are non-mobile blood components and include tissues, membrane receptors, interstitial proteins, fibrin proteins, collagens, platelets, endothelial cells, epithelial cells and their associated membrane and membraneous receptors, somatic body cells, skeletal and smooth muscle cells, neuronal components, osteocytes and osteoclasts and all body tissues especially those associated with the circulatory and lymphatic systems.

Mobile blood components: Mobile blood components are blood components that do not have a fixed situs for any extended period of time, generally not exceeding 5, more usually one minute. Mobile blood components include soluble blood proteins such as immunoglobulins, serum albumin, ferritin, transferrin and the like.

Wound Healing Agents: Wound healing agents are agents that promote wound healing. Wound healing agents include integrins, cell adhesion molecules such as ICAM, ECAM, ELAM and the like, antibiotics, growth factors such as EGF, PDGF, IGF, bFGF, aFGF and KGF, fibrin, thrombin, RGD peptides and the like.

Antiproliferatives: Antiproliferatives include antimetabolites, topoisomerase inhibitors, folic acid antagonists like methotrexate, purine antagonists like mercaptopurine, azathioprine, and pyrimidine antagonists like fluorouracil, cytarabine and the like.

Antioxidants: Antioxidants are agents that prevents oxidative damage to tissue and include aspartate, orotate, tacophenol derivative (vitamin E), and free radical scavengers such as SOD, glutathione and the like.

Mammalian cells are continuously exposed to activated oxygen species such as superoxide, hydrogen peroxide, hydroxyl radical, and singlet oxygen. These reactive oxygen intermediates are generated in vivo by cells in response to aerobic metabolism, catabolism of drugs and other xenobiotics, ultraviolet and x-ray radiation, and the respiratory burst of phagocytic cells (such as white blood cells) to kill invading bacteria such as those introduced through wounds. Hydrogen peroxide, for example, is produced during respiration of most living organisms especially by stressed and injured cells.

Active oxygen species can injure cells. An important example of such damage is lipid peroxidation which involves the oxidative degradation of unsaturated lipids. Lipid peroxidation is highly detrimental to membrane structure and function and can cause numerous cytopathological effects. Cells defend against lipid peroxidation by producing radical scavengers such as superoxide dismutase, catalase, and peroxidase. Injured cells have a decreased ability to produce radical scavengers. Excess hydrogen peroxide can react with DNA to cause backbone breakage, produce mutations, and alter and liberate bases. Hydrogen peroxide can also react with pyrimidines to open the 5,6-double bond, which reaction inhibits the ability of pyrimidines to hydrogen bond to complementary bases, Hallaender et al. (1971). Such oxidative biochemical injury can result in the loss of cellular membrane integrity, reduced enzyme activity, changes in transport kinetics, changes in membrane lipid content, and leakage of potassium ions, amino acids, and other cellular material.

Antioxidants have been shown to inhibit damage associated with active oxygen species. For example, pyruvate and other alpha-ketoacids have been reported to react rapidly and stoichiometrically with hydrogen peroxide to protect cells from cytolytic effects, O'Donnell-Tormey et al., J. Exp. Med., 165, pp. 500-514 (1987).

Anti-Infective Agents: Anti-infective agents are agents that inhibit infection and include anti-viral agents, anti-fungal agents and antibiotics.

Anti-Viral Agents: Anti-viral agents are agents that inhibit virus and include vidarabine, acyclovir and trifluorothymidine.

Anti-Fungal Agents: Anti-fungal agents are agents that inhibit fungal growth. Anti-fungal agents include anphoterecin B, myconazole, terconazole, econazole, isoconazole, thioconazole, biphonazole, clotrimazole, ketoconazole, butaconazole, itraconazole, oxiconazole, phenticonazole, nystatin, naphthyphene, zinoconazole, cyclopyroxolamine and fluconazole.

Antibiotics: Antibiotics are natural chemical substances of relatively low molecular weight produced by various species of microorganisms, such as bacteria (including Bacillus species), actinomycetes (including Streptomyces) and fungi, that inhibit growth of or destroy other microorganisms. Substances of similar structure and mode of action may be synthesized chemically, or natural compounds may be modified to produce semi-synthetic antibiotics. These biosynthetic and semi-synthetic derivatives are also effective as antibiotics. The major classes of antibiotics are (1) the beta-lactams, including the penicillins, cephalosporins and monobactams; (2) the aminoglycosides, e.g. gentamicin, tobramycin, netilmycin, and amikacin; (3) the tetracyclines; (4) the sulfonamides and trimethoprim; (5) the fluoroquinolones, e.g. ciprofloxacin, norfloxacin, and ofloxacin; (6) vancomycin; (7) the macrolides, which include for example, erythromycin, azithromycin, and clarithromycin; and (8) other antibiotics, e.g., the polymyxins, chloramphenicol and the lincosamides.

Antibiotics accomplish their anti-bacterial effect through several mechanisms of action which can be generally grouped as follows: (1) agents acting on the bacterial cell wall such as bacitracin, the cephalosporins, cycloserine, fosfomycin, the penicillins, ristocetin, and vancomycin; (2) agents affecting the cell membrane or exerting a detergent effect, such as colistin, novobiocin and polymyxins; (3) agents affecting cellular mechanisms of replication, information transfer, and protein synthesis by their effects on ribosomes, e.g., the aminoglycosides, the tetracyclines, chloramphenicol, clindamycin, cycloheximide, fucidin, lincomycin, puromycin, rifampicin, other streptomycins, and the macrolide antibiotics such as erythromycin and oleandomycin; (4) agents affecting nucleic acid metabolism, e.g., the fluoroquinolones, actinomycin, ethambutol, 5-fluorocytosine, griseofulvin, rifamycins; and (5) drugs affecting intermediary metabolism, such as the sulfonamides, trimethoprim, and the tuberculostatic agents isoniazid and para-aminosalicylic acid. Some agents may have more than one primary mechanism of action, especially at high concentrations. In addition, secondary changes in the structure or metabolism of the bacterial cell often occur after the primary effect of the antimicrobial drug.

Anti-Cancer Agents: Anti-cancer agents are natural or synthetic molecules which are effective against one or more forms of cancer. This definition includes molecules which by their mechanism of action are cytotoxic (anti-cancer chemotherapeutic agents), those which stimulate the immune system (immune stimulators) and modulators of angiogenesis. The outcome in either case is the slowing of the growth of cancer cells.

Anti-cancer therapy include radioactive isotopes such as ³²P used in the treatment of polycythemia vera and in chronic leukemia. Radioactive phosphorus has a biological half-life of about 8 days in humans. It emits beta rays that exert a destructive effect on the rapidly multiplying cells. ³²P is usually administered in doses of about 1 mc daily for 5 days. Either the oral or intravenous route may be used and the doses are not greatly different. Radioactive iodine ¹³¹I, radioactive gold ¹⁹⁸Au, and other isotopes are not as useful as ³²P. Nevertheless, ¹³¹I has some limited applications in metastatic thyroid carcinoma. Other radioactive isotopes can be used with our technology either as complexes of radioactive metal such as ⁵¹Cr, ⁵²Mn, ⁵²Mg, ⁵⁷Ni, ⁵⁵Co and ⁵⁶P, ⁵⁵Fe, ¹⁰³Pd, ¹⁹²Ir, ⁶⁴Cu and ⁶⁷Cu or as chelates of these metals using bifunctional chelating agents like (BFCs), 6-[p-(bromoacetamido)benzyl]-1,4,8,11-tetraazacyclotetradecane-1,4,8,11-tetraacetic acid (BAT), 6-[p-(isothiocyanato)benzyl]-1,4,8,11-tetraazacyclotetradecane-1,4,8,11-tetraacetic acid (SCN-TETA), 4-[(1,4,8,11-tetraazacyclotetradec-1-yl) methyl]benzoic acid (CPTA), and 1-[(1,4,7,10,13-pentaazacyclopentadec-1-yl)methyl]benzoic acid (PCBA).

Numerous drugs fall into the category of chemotherapeutic agents useful in the treatment of neoplastic disease that are amenable to the embodiment of this application for local drug delivery and retention of the modified drug substance at the tumor site. Such agents derivitized with this technology can include antimetabolites such as metotrexate (folic acid derivatives), fluoroaucil, cytarabine, mercaptopurine, thioguanine, petostatin (pyrimidine and purine analogs or inhibitors), a variety of natural products such as vincristine and vinblastine (vinca alkaloid), etoposide and teniposide, various antibiotics such as miotomycin, plicamycin, bleomycin, doxorubicin, danorubicin, dactomycin; a variety of biological response modifiers including interferon-alpha; a variety of miscellaneous agents and hormonal modulators including cisplatin, hydroxyurea, mitoxantorne, procarbozine, aminogultethimide, prednisone, progestins, estrogens, antiestorgens such as tamoxifen, androgenic steroids, antiadrogenic agents such as flutamide, gonadotropin releasing hormones analogs such as leuprolide, the matrix metalloprotease inhibitors (MMPIs) as well as anti-cancer agents including Taxol (paclitaxel) and related molecules collectively termed taxoids, taxines or taxanes.

Included within the definition of “taxoids” are various modifications and attachments to the basic ring structure (taxoid nucleus) as may be shown to be efficacious for reducing cancer cell growth and which can be constructed by organic chemical techniques known to those skilled in the art.

Chemotherapeutics include podophyllotoxins and their derivatives and analogues. Another important class of chemotherapeutics useful in this invention are camptothecins.

Another preferred class of chemotherapeutics useful in this invention are the anthracyclines (adriamycin and daunorubicin).

Another important class of chemotherapeutics are compounds which are drawn from the following list: Taxotere, Amonafide, Illudin S, 6-hydroxymethylacylfulvene Bryostatin 1, 26-succinylbryostatin 1, Palmitoyl Rhizoxin, DUP 941, Mitomycin B, Mitomycin C, Penclomedine, angiogenesis inhibitor compounds, Cisplatin hydrophobic complexes such as 2-hydrazino-4,5-dihydro-1H-imidazole with platinum chloride and 5-hydrazino-3,4-dihydro-2H-pyrrole with platinum chloride, vitamin A, vitamin E and its derivatives, particularly tocopherol succinate.

Other compounds useful in the invention include: 1,3-bis(2-chloroethyl)-1-nitrosurea (“carmustine” or “BCNU”), 5-fluorouracil, doxorubicin (“adriamycin”), epirubicin, aclarubicin, Bisantrene (bis(2-imidazolen-2-ylhydrazone)-9,10-anthracenedicarboxaldehyde, mitoxantrone, methotrexate, edatrexate, muramyl tripeptide, muramyl dipeptide, lipopolysaccharides, vidarabine and its 2-fluoro derivative, resveratrol, retinoic acid and retinol, carotenoids, and tamoxifen.

Other chemotherapeutic agents useful in the application of this invention include: Decarbazine, Lonidamine, Piroxantrone, Anthrapyrazoles, Etoposide, Camptothecin, 9-aminocamptothecin, 9-nitrocamptothecin, camptothecin-11 (“Irinotecan’), Topotecan, Bleomycin, the Vinca alkaloids and their analogs [Vincristine, Vinorelbine, Vindesine, Vintripol, Vinxaltine, Ancitabine], 6-aminochrysene, and Navelbine.

Other compounds useful in the application of the invention are mimetics of taxol, eleutherobins, sarcodictyins, discodermolides and epothiolones.

RGD Peptides: The RGD peptide for conjugation to tissues or fixed endogenous proteins in accordance with the present invention includes a sequence of amino acids, preferably naturally occurring L-amino acids and glycine, having the following formula:

R₁-Arg-Gly-Asp-R₂

In this formula, R₁ and R₂ represent an amino acid or a sequence of more than one amino acid or a derivatized or chemically modified amino acid or more than one derivatized or chemically modified amino acids.

Delivery Devices: Delivery devices are devices useful for local delivery of therapeutic agents. Delivery devices include catheters, syringes, trocars and endoscopes.

Reactive Entities: Reactive entities are entities capable of forming a covalent bond. Such reactive agents are coupled or bonded to a therapeutic agent of interest. Reactive entities will generally be stable in an aqueous environment and will usually be carboxy, phosphoryl, or convenient acyl group, either as an ester or an anhydride, or an imidate, thereby capable of forming a covalent bond with an amino group at the target site to form an amide or amide derivative. For the most part, the esters will involve phenolic compounds, or be thiol esters, alkyl esters, phosphate esters, or the like.

While the reactive entity is usually chosen to react with an amino group at the target site, other reactive functionalities at the target site may be exploited. For example, the reactive functionality may comprise various phosphinyl or phosphonyl derivatives for the bonding to available hydroxyl functions at the target site or may comprise an imine, thioimine or disulfide for bonding to thiol residues.

Reactive Functionalities: The reactive functionalities available on vascular proteins for covalent bond formation with the reactive group are primarily amino, carboxyl and thiol groups. While any of these may be used as the target for the reactive entity, for the most part, bonds to amino groups will be employed, particularly with the formation of amide bonds.

To form amide bonds, one may employ a wide variety of active carboxyl groups as the reactive functional group of the bifunctional molecule, particularly esters, where the hydroxyl group is physiologically acceptable at the levels required. While a number of different hydroxyl groups may be employed, the most convenient will be N-hydroxysuccinimide and N-hydroxy sulfosuccinimide, although other alcohols, which are functional in the vascular environment may also be employed. In some cases, special reagents find use such as diazo, azido, carbodiimide, anhydride, hydrazine, or thiol groups, depending on whether the reaction is in vivo or in vitro, the target, the specificity of the reactive entity, and the like.

IC50: Concentration of an enzyme inhibitor at which 50% of the enzymatic activity is inhibited.

Protective Groups: Protective groups are chemical moieties utilized to protect reactive entities from reacting with themselves. Various protective groups are disclosed in U.S. Pat. No. 5,493,007 which is hereby incorporated by reference. Such protective groups include acetyl, fluorenylmethyloxycarbonyl (FMOC), t-butyloxy carbonyl (BOC), benzyloxycarbonyl (CBZ), and the like.

Linking Groups: Linking groups are chemical moieties that link or connect reactive entities to therapeutic agents. Linking groups may comprise one or more alkylene, alkyleneoxy, alkenylene, alkynylene or amino group substituted by alkyl groups; cycloalkylene groups, polycyclic groups, aryl groups, polyaryl groups, substituted aryl groups, heterocyclic groups, and substituted heterocyclic groups. Linking group will have from 2-100, more usually from 2-18, preferably from 6-12 atoms in the chain, particularly carbon, oxygen, phosphorous and nitrogen, more particularly carbon and oxygen.

### DETAILED DESCRIPTION

Taking into account these definitions, in its first aspect, the invention is directed to the local delivery of therapeutic agents which have been modified with reactive entities so that they will covalently react and bond in vivo with reactive functionalities onto a fixed blood component and provide increased half lifes for the therapeutic agents.

The derivatized therapeutic agent of the present invention will, for the most part, have the following formula: X—Y-Z wherein:

X is selected from the group consisting of wound healing agents, antibiotics, anti-inflammatories, antioxidants, antiproliferatives, immunosuppressants, anti-infective and chemotherapeutic agents;

In the formula, Y is a linking group of from 0-30, more usually of from 2-12, preferably of from 4-12 atoms, particularly carbon, oxygen, phosphorous and nitrogen, more particularly carbon and oxygen, where the oxygen is preferably present as oxy ether, where Y may be alkylene, oxyalkylene, or polyoxyalkylene, where the oxyalkylene group has from 2-3 carbon atoms, and the like. A linking group of O atoms is preferred when it is desired to place X as close to Z as possible;

In the formula, Z is a reactive entity, such as carboxy, carboxy ester, where the ester group is of 1-8, more usually 1-6 carbon atoms, particularly a physiologically acceptable leaving group which activates the carboxy carbonyl for reaction with amino groups in an aqueous system, e.g. N-hydroxysuccinimide (NHS), N-hydroxysulfosuccinimide, (sulfo-NHS), maleimide-benzoyl-succinimide (MBS), gamma-maleimido-butyryloxy succinimide ester (GMBS) and maleimidopropionic acid (MPA), N-hydroxysuccinimide isocyanate, isothiocyanate, thiolester, thionocarboxylic acid ester, imino ester, mixed anhydride, e.g. carbodiimide anhydride, carbonate ester, etc. and the like.

The reactive functionalities which are available on proteins for covalently bonding to the chemically reactive entity of the derivatized therapeutic agent are primarily amino groups, carboxyl groups and thiol groups. While any of these may be used as the target of the chemically reactive entity on the therapeutic agent, for the most part, bonds to amino groups will be employed, particularly with formation of amide bonds. To form amide bonds, one may use as a chemically reactive group a wide-variety of active carboxyl groups, particularly esters, where the hydroxyl moiety is physiologically acceptable at the levels required. While a number of different hydroxyl groups may be employed, the most convenient will be N-hydroxysuccinimide, (NHS) and N-hydroxy sulfosuccinimide, (sulfo-NHS), maleimide-benzoyl-succinimide (MBS), gamma-maleimido-butyryloxy succinimide ester (GMBS) and maleimidopropionic acid (MPA), although other alcohols, which are functional in an aqueous medium such as blood, may also be employed. In some cases, special reagents find use, such as azido, diazo, carbodiimide, anhydride, hydrazine, dialdehydes, thiol groups, or amines to form amides, esters, imines, thioethers, disulfides, substituted amines, or the like. Usually, the covalent bond which is formed should be able to be maintained during the lifetime of the blood or tissue component, unless it is intended to be a release site. In the preferred embodiments of this invention, the functional group on this protein will be a thiol group and the chemically reactive group will be a maleimido-containing group such as GMBA or MPA. GMBA stands for gamma-maleimide-butyrylamide.

The manner of producing the derivatized therapeutic agents of the present invention will vary widely, depending upon the nature of the various elements comprising the molecule. The synthetic procedures will be selected so as to be simple, provide for high yields, and allow for a highly purified product. Normally, the chemically reactive group will be created as the last stage, for example, with a carboxyl group, esterification to form an active ester will be the last step of the synthesis. Methods for the production of derivatized therapeutic agents of the present invention are described in examples 1-6. Each therapeutic agent selected to undergo the derivatization with a linker and a reactive agent will be modified according to the following criteria: if a carboxylic group, not critical for the retention of pharmacological activity is available on the original molecule and no other reactive functionality is present on the molecule, then the carboxylic acid will be chosen as attachment point for the linker-reactive entity modification. If no carboxylic acids are available, then any other functionalities not critical for the retention of pharmacological activity will be selected as attachment point for the linker-reactive entity modification. If several functionalities are available on a therapeutic agent, a combination of protecting groups will be used in such a way that after addition of the linker/reactive entity and deprotection of all the protected functional groups, retention of pharmacological activity is still obtained. If no reactive functionalities are available on the therapeutic agent, synthetic efforts will allow for a modification of the original parent drug in such a way that retention of biological activity and retention of receptor or target specificity is obtained.

The chemically reactive entity is at a site, so that when the therapeutic agent is bonded to the fixed blood component, the therapeutic agent retains a substantial proportion of the parent compound's inhibitor activity.

The derivatized therapeutic agent of the present invention will generally have substantially lower IC₅₀'s generally in the range of about 0.5-0.01 of the IC₅₀ of the parent molecule. Desirably, the IC₅₀ should be not less than 0.05, preferably not less than about 0.1. In view of the varying IC₅₀'s, the amount of the derivatized therapeutic agent administered will also vary.

The determination of the nature and length of the linker will be performed through an empirical optimization phase and will be measured by the retention or the loss of biological activity. For instance, with a given inhibitor enzyme interactions, an iteration of the modification of the nature and the length of the linker and a measure of the biological enzymatic activity may be necessary to determine the most favored linker length and nature. Preferably a short hydrophilic 4-12 atom linker easily synthesized will be favored to start the iteration process.

In the case of radiolabeled therapeutic agents, a minimum distance from the target has to be respected based on the nature of the isotope and its penetration. The length and nature of the linkers are not as important as they are for an enzyme inhibitor combination. For instance an isotope that emits a beta rays like ³²P should be positioned within 5 mm from the target to have maximum efficiency (99%) with limited or no effect coming from a small change on the nature and length of the linker.

The reactive entities have to be chosen in such a way that most of the reactive entity reacts with the reactive functionality in the shortest amount of time for an in vivo application. Some surgical applications require a maximum incubation time of three minutes, due to biological constrains (interruption of arterial blood flow for instance). Some other surgical intervention like vascular grafting which is performed ex vivo may take several minutes or hours if the graft tissue is kept under appropriate conditions of conservation. The reactive entities may thus be different for the type of application based on their rate of reaction with the reactive functionalities. Preferably, 1-30% of the molecule added will bond to the target reactive functionality in vivo within a 3 minutes incubation window. More preferably 10-90% of the reactive entity will bond to the reactive functionality in vivo within a 3 minute window. Most preferably, the reaction will be over within 5 minutes. For an ex vivo loading, 10% of the molecule will add to the reactive functionality within a 3 minute window. Preferably 10-50% of the reactive entity will add to the reactive functionality within a 3 minute window. Most preferably, the reaction will be over within 15 minutes.

Modified therapeutic agents are infused either via standard balloon catheters, using pressure or iontophoresis to produce an efficient distribution of the infused drug substance, or introduced via standard techniques of infusion, lavage or topical application so that the drug is locally delivered to the site of interest. Drugs are modified with appropriate connectors to retain activity when bound to exposed proteins in the vessel wall. In vivo covalent attachment is achieved through the use of either non-specific NHS or other crosslinking agents or through the use of site specific covalent affinity probes to specific proteins. Once bound, the modified drugs retain biological activity. The covalent attachment of the therapeutic agents provides a long lasting effect superior in time to existing methods or local drug delivery.

The technology described herein finds application to a broad range of therapeutic uses including the following:

1. RGD Peptides

A. RGD Peptides for Improved Wound Healing

RGD (Arg-Gly-Asp)-containing peptides and proteins such as Osteopontin have been recognized to accelerate wound healing in animal models. The mechanism by which these peptides are thought to work is through stimulation of fibroblast and epithelial cell migration and adhesion into the wound. The RGD containing peptides bind to a defined receptor, the alpha(v)beta3 integrin receptor on these cells which stimulates attachment and migration. RGD containing peptides have failed to improve wound healing, i.e. acceleration or increased strength, in human clinical trials. These peptides most likely failed to work because the residence time in the dermal ulcer/wound was too brief and/or because of lack of attachment to fixed structures in the wound substratum.

An example of an RGD containing peptide useful in the present invention is the following:

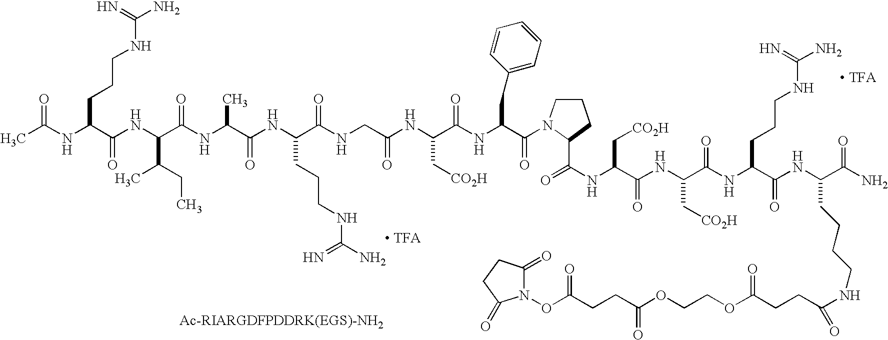

An RGD containing peptide, such as those that are the subject of the current application, could be covalently coupled to the wound substratum using reactive chemistry and, in combination with the appropriate spacer/linker, be available for a more prolonged but correct presentation to wound healing elements than are conventionally applied RGD containing peptides. Binding and attachment to cellular wound healing elements such as fibroblasts and epithelial cells through the alpha(v)beta3 receptor is not altered by the chemistry employed in the current application.

The drug product described here could be applied topically to skin wounds and incisions including burns, dermal ulcers, trophic ulcers, diabetic ulcers, surgical incisions, skin graft donor and acceptor sites, surgical flap repairs, excision biopsy sites, fistulae, fissures, as well as sites of gastrointestinal ulceration in the stomach, duodenum, colon or rectum. The agent could also be applied to sites of intestinal or other visceral anastamosis, or peritoneal, pleural, or mucosal surfaces to stimulated repair and healing. The agent would be applied to the raw surface after adequate debridment either physically be irrigation or cleaning, or following enzymatic preparation and cleaning with agents such as hyaluronidase, papain, etc.

The drug could be applied to a cleaned and slightly moist wound surface either in an aqueous solution, in DMSO, as a powder, an aerosolized mist, ointment, lotion, emulsion, or as a film or as a component of a bandage or wrap. Unreacted drug may be removed after a period of time such as 5-20 mins of application. The wound should be infection free and kept clean and most likely bandaged with wet to dry dressings post application.

B. RGD Peptides as Anti-Angiogenic Agents

Tumor metastasis is characterized by a series of steps involving interaction of various host cells (endothelial cells, platelets, lymphocytes) and an extracellular matrix such as collagens I and IV, fibronectin, laminin and sulfated glycosaminoglycans. The interactions between cells and components of the extracellular matrix are regulated by cell surface receptors called integrins. Some synthetic peptides derived from adhesion molecules that are present in the extracellular matrix have been shown to modulate the mechanism involved in the metastasizing function of tumor cells. For instance several integrins recognize the amino acid sequence RGD which mediates the adhesion of normal and tumor cells to components of the extracellular matrix.

Angiogenesis is a multistep process involving matrix degradation, cellular proliferation and migration and recolonization in which capillary endothelial cells sever their normal cell cell attachment, migrate through the extracellular matrix and reform cell cell attachment to create new capillary.

C: RGD Peptides as Anti-Restenosis Agents

Restenosis is the result of a complex vascular wound healing that occurs in most of the patients who undergo angioplasty therapy. Recent studies suggest that a cellular proliferative process leading to intimal hyperplasia and the remodeling of the vessel are responsible for the phenomenon of restenosis also described as the renarrowing of a vessel after angioplasty. Srivatsa et al. (Cardio Res 1997 36:408-428) demonstrated that RGD peptidomimetics. limits neointimal hyperplasia and lumen stenosis following deep porcine coronary arterial injury. They also noticed that sustained pharmacologic blockade of alpha(v)beta(3) beyond 14 days post injury is required to achieve maximal anti-restenosis efficacy. Spelian, et al. (Circulation 1998 97:1818-27) showed that local delivery of cyclic RGD peptides to the adventitial surface of balloon-injured rat carotid arteries led to a significant reduction in smooth muscle cells migration and reduction in neointimal thickening at 14 days after balloon injury. An RGD containing peptide, such as those that are the subject of the current application, could be covalently coupled to the site where the angioplasty was performed and the sustained pharmalogic blockade of the alpha(v) beta(3) receptor should be achieved beyond 14 days thus allowing maximal anti-restenosis efficacy.

The RGD peptide for conjugation to tissues or fixed endogenous proteins in accordance with the present invention includes a sequence of amino acids, preferably naturally occurring L-amino acids and glycine, having the following formula:

R₁-Arg-Gly-Asp-R₂

In this formula, R₁ and R₂ represent an amino acid or a sequence of more than one amino acid or a derivatized or chemically modified amino acid or more than one derivatized or chemically modified amino acids.

In a specific embodiment, R₁ represents XY(Z)ₙ, in which X, Y and Z independently represent an amino acid; and n represents 0 or 1; R₂ represents OH or NH₂; or any amino acid; or a sequence of more than one amino acid or a derivatized or chemically modified amino acid. In a specific embodiment, R₂ represents an amino acid other than serine, threonine or cysteine or the amide thereof wherein the amino acid is rendered a carboxyamide. In another specific embodiment, R₂ is more than one amino acid, the first amino acid in the sequence, which is attached to aspartic acid, being other than serine, threonine or cysteine, or the amide of any free carboxyl groups wherein R₂ includes a derivatized or chemically modified amino acid.

In a preferred embodiment, R₂ includes a linking group having a chemically reactive group which covalently bonds to reactive functionalities or proteins and R₁ includes a protective group to prevent the chemically reactive group of R₂ from reacting with R₁. In another embodiment, R₁ includes a linking group having a chemically reactive group which covalently bonds to reactive functionalities on proteins and R₂ includes a protective group to prevent the chemically reactive group of R₁ from reacting with R₂.

In yet another embodiment, both R₁ and R₂ include a linking group having a chemically reactive entity which covalently bonds to functionalities on fixed proteins. In this embodiment, the linking groups may be similar or different.

In the RGD peptide of this invention, I, R₁ and R₂ may include any amino acid or sequence thereof. The amino acids are preferably naturally occurring. The most common naturally-occurring amino acids are shown in Table 1:

TABLE I
NATURAL AMINO ACIDS AND THEIR ABBREVIATIONS

| | 3-Letter | 1-Letter |
|---|---|---|
| Name | Abbreviation | Abbreviation |
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Try | W |
| Tyrosine | Tyr | Y |
| Valine | Val | V |

However, R₁ and R₂ in the RGD peptide of this invention are not limited to the 20 natural-amino acids. In other embodiments, R₁ and R₂ can be D-amino acids, non-classical amino acids or cyclic peptides or peptidomimetics (chemical peptide analogs). Non-classical amino acids include but are not limited to the D-isomers of the common amino acids, α-amino isobutyric acid, 4-aminobutyric acid, hydroxyproline, sarcosine, citrulline, cysteic acid, t-butylglycine, t-butylalanine, phenylglycine, cyclohexylalanine, β-alanine, designer amino acids such as β-methyl amino acids, Cα-methyl amino acids, Nα-methyl amino acids, and amino acid analogs in general.

Furthermore, the Arg and/or Asp in the RGD sequence can be the D (dextrarotary) or L (levorotary) amino acid.

When R₁ and/or R₂ are a sequence of amino acids, there is no necessary limitation on the number of amino acids in the sequence(s). Accordingly, the polypeptide for conjugation to fixed blood proteins can be any size, and encompasses what might otherwise be called an oligopeptide, a protein, an organic molecule or a polymer such as polyethylene glycol. Preferably, the polypeptide will have no more than about 1,000 amino acids.

The polypeptide may be prepared by methods that are known in the art. For example, in brief, solid phase peptide synthesis consists of coupling the carboxyl group of the C-terminal amino acid to a resin and successively adding N-alpha protected amino acids. The protecting groups may be any known in the art. Before each new amino acid is added to the growing chain, the protecting group of the previous amino acid added to the chain is removed. The coupling of amino acids to appropriate resins is described by Rivier et al., U.S. Pat. No. 4,244,946. Such solid phase syntheses have been described, for example, by Merrifield, 1964, J. Am. Chem. Soc. 85:2149; Vale et al., 1981, Science 213:1394-1397; Marki et al., 1981, J. Am. Chem. Soc. 103:3178 and in U.S. Pat. Nos. 4,305,872 and 4,316,891.

Derivatives of RGD peptides and their analogs which can conjugate with proteins and other fixed blood proteins are prepared as is known in the art by the use of linking groups having chemically reactive groups which covalently bond to reactive functionalities on proteins.

2. Gastrointestinal Ulcer Treatment

Integrins, growth factors and cell adhesion molecules may be modified using the NHS/linker technology disclosed here so that they will covalently bond to the base of an esophageal, gastric or duodenal ulcer when applied topically to the lesion via an endoscope. Continued high levels of topically applied growth factors such as, but not limited to, GF, PDGF, IGF, bFGF, aFGF and KGF, and integrins and cell adhesion molecules such as those mentioned above will stimulate reepithelialization and ulcer healing. NSAIDS, steroids and other antiinflammatory drugs could also be modified by our technology to produce ulcer healing.

In addition, antibacterials or bacteriostatic agents such as bismuth sulphate designed to kill or inactivate Heliobacter pylorii could be applied to the base of peptic ulcers in order to achieve persistent and high local concentrations of these therapies in the region of the ulcer to aid healing. H. pylorii is recognized as an etiological factor in peptic ulcers and its continued presence in the ulcer is recognized to interfere with healing.

3. Intraoperative Administration to Prevent Adhesions

Intraabdominal and intrathoracic surgery is often complicated by adhesions which develop within weeks to months postoperatively. These adhesions may remain undetected and of no consequence; however, often months to years postoperatively they may cause problem with the function of an organ or viscus, such as the intestines, or cause intractable pain requiring reoperation. Using antiinflammatory peptides and drugs such as inhibitors of cell and matrix adhesion molecules coupled via spacers to NHS, topically applied during surgery to key sites that are prone to adhesions such as small or large intestinal anastomoses, adhesions can be prevented.

4. Intraoperative Administration to Prevent Bleeding

Fibrin, thrombin or tissue factor peptides, and fragments of these and other procoagulant proteins and drugs can be attached to sites of bleeding in the body using NHS/linker technology to effect hemostasis to prevent bleeding. These agents can be topically administered to sites of bleeding during open or laparascopic or other minimally invasive surgery, including but not limited to arthroscopy, thorascopic, or culdoscopic and endoscopic surgery. These agents could also be used to prevent bleeding post arteriotomy, or locally at sites following arterial puncture with catheters such as angiographic, angioplasty or hemodialysis or hemoperfusion catheters to reduce the risk of post puncture hemorrhage or intrauterine for post partum hemmorhage or dysfunctional uterine bleed. These agents could also be of great value to prevent potentially serious hemorrhage intrathoracically post cardiac surgery, intracranially following neurosurgery, or after repair of an aneurysm either intracranially, intrathoracically, or intraabdominally.

5. Opthalmic Surgery

The technology of this invention may be used to affect retention and the local effect of antiproliferatives to prevent and/or minimize adhesions in opthalmic surgery. In particular, it could be used during anterior chamber surgery for glaucoma to prevent postoperative growth of a proliferative tissue in the region of the trabecular meshwork or anterior chamber resorptive apparatus. In addition topical use of protease inhibitors derivatized with NHS esters could be utilized to inhibit bacterial damage to the sclera as well as directly injected into the retinal to inhibit angiogenesis and macula degeneration.

6. Urology

In urological procedures, postoperative hemorrhage is common and the procoagulants mentioned above when coupled, via linker technology, to reactive NHS esters can be applied topically via a cystoscope or in an open operative field to sites of potential hemorrhage. The technology of the invention may be applied postoperatively for the prevention of hemorrhage following prostate surgery including but not limited to TURP or open prostatectomy, polyp removal, bladder cancer removal or partial bladder resection. The technology can be used to apply bacteriostatic or bactericidal agents perioperatively via a cystoscope or postoperatively via a Mey catheter and bladder irrigation to prevent or treat infection. Antiinflammatory agents can be similarly applied to prevent postoperative urethral stricture formation.

7. Endoscopy

The same technology could be used in the prevention and treatment of esophageal stricture formation following esophageal surgery or due to benign causes such as Barrett's esophagus or severe reflux esophagitis. Endoscopy application of phosphodiesterase inhibitors such as methylxanthines (pentoxifyline, aminophylline, theophylline and related derivatives) as well as the local covalent linkage of selected anti-inflammatory agents may be useful in managing patients in acute asthmatic attack or cystic fibrosis.

8. Biliary Surgery

NHS/linker coupled anti-inflammatory agents, or bacteriostatic/bactericidal agents such as those described above can be applied topically to sites of anastomosis/resection during open or laparoscopic biliary surgery to prevent adhesions, strictures and infection.

9. Colonoscopic Use

The technology may be utilized colonoscopically in the management of inflammatory bowel disease (IBD) to apply and sustain high local concentrations of select small molecule antiinflammatory drugs such as steroids, NSAIDS and aspirin. Selectins and inhibitors of cell adhesion and antiproliferatives can be applied topically to the inflamed section/sections of the colon affected by Crohn's disease or Ulcerative Colitis in order to accelerate healing, reduce inflammation, and prevent stricture formation.

10. Spinal Cord and Peripheral Nerves

Topical application of TRH or a neurotrophic growth factor such as NGF, BNDF, CNTF, PTN, MK, or NTII coupled via a linker to NHS, during open surgical repair of either a transected or injured spinal cord, or damaged or transected peripheral nerve can be undertaken using this technology to improve nerve regeneration.

11. Pulmonary

The technology can be used to apply anti-inflammatory and antibacterial agents to sites of biopsy or stricture in the airway when identified and visualized endobronchially. Similarly, hemostasis can be achieved or aided by the topical application post-biopsy of procoagulant factors/peptides attached via linkers to NHS esters.

Topical application of selected thrombin inhibitors attached via linkers to NHS esters may aid in the inhibition of small cell lung carcinoma and act as a an adjunctive therapy to surgical resection procedures.

12. Dental

Procoagulant and antibacterial factors can be applied to tooth sockets postextraction to prevent bleeding and infection. Local application of selected antibiotics such as tetracyclin can result in the inhibition of proteases that are associated with gingivitis and gum deterioration by permanently adhering the drug for prolonged periods of time.

13. ENT Surgery

In a similar fashion these procoagulant and antibacterial drugs coupled to NHS via linkers can be applied topically to head and neck sites during surgery such as tumor resection to prevent bleeding and infection. The procoagulant coupled NHS agents can be applied topically to nosebleeds, especially severe posterior septal bleeds which are prone to recurrence and are difficult to access and control.

14. Intratumor

Slow and sustained intratumor release of anticancer agents is possible using this NHS/linker technology in order to achieve sustained killing of tumor cells over a period of days to weeks. This can be achieved by intratumor injection of antiproliferative drugs coupled via linker technology to reactive NHS esters. The NHS coupled drugs will react following injection with stomal and cellular elements of the tumor and can be released slowly into the tumor from these anchored sites by breakdown of the attached site.

15. Catheter-Based Radiotherapy

As a replacement for radioactive implants or as a substitute for radioactive injection of liquid for multiple applications. ¹⁹²Ir and ³²P are known to reduce coronary restenosis in patients with previous restenosis. The intracoronary radiotherapy has been shown to reduce the intimal hyperplasia that is part of restenosis. ¹³⁷Cs, ⁸⁹Sr and ⁹⁰Sr, ³²P, Iodine ¹²⁵I and ¹³¹I, ¹⁹²Ir, ⁹⁰Y, ⁹⁰Y/Sr, bismuth, radium are used internal therapy of various forms of cancers including those in the mouth, lip, breast, anus, vagina, thyroid, bone marrow, lungs and prostate. In most cancer cases, the radiotherapy has been shown to shrink the tumor or reduce the risk of spreading before and during surgery. Internal radiotherapy is also given to kill off any tiny amounts of the tumor that may have been left after surgery.

All these isotopes could be covalently attached to the tumor or in the vicinity of the tumor itself for either palliative, curative and radical treatments or as an adjuvant to other therapies.

For instance ³²P can be covalently attached to tissue or membrane proteins through the use of an NHS phosphodiester or triester.

Other radioisotopes can be used with our technology either as complexes of radioactive metal such as ⁵¹Cr, ⁵²Mn, ⁵²Mg, ⁵⁷Ni, ⁵⁵Co and ⁵⁶P, ⁵⁵Fe, or as chelates of these metals. Preferred radioactive isotopes are beta ray and gamma ray emitters.

16. Immuno-Suppressant Activity

A variety of immuno-suppressant agents such as cyclosporin and derivatives, corticosteroids, sulfasalazine, thalidomide, methotrexate, OKT3, peptide-T, or agents that inhibit T-cell activation or adhesion would be useful to locally apply to organs prior to transplantation to mask immune responsiveness and organ rejection. Such agents could be applied locally at the time of tissue harvest (e.g. heart, lung, liver harvest) or immediately prior to restitution of blood flow in the recipient. Such immuno-suppressant agents would prevent the recognition of foreign antigen from the donor tissue that would facilitate short term acceptance and facilitate longer term ability for the host to accommodate the transplanted organ.

17. Intra-Cardiac Delivery

Slow and sustained presence of selected drugs into the heart is possible using this NHS/linker technology in order to achieve sustained presence of selected drugs over a period of days to weeks. Such agents could be delivered to the heart by pericardial delivery catheter, percutaneous delivery catheter or by direct injection into the epicardium, myocardium or endocardium during open chest or open heart surgical procedures. Such drugs delivered in this matter could include NHS derivatives of anti-arrhythmic agents such as disopyramide, quinidine, amiodarone to control cardiac rate and rhythm disturbances; antibiotics or anti inflammatory agents such as corticosteroids into the pericardial space to control pericardititis; growth factors such as VEGF or FGF to induced topical revascularization, or inotropic agents such as methylxanthines, digitalis or phosphodiesterase inhibitors to improve contractility and ejection fraction in the failing heart.

18. Intra-Medullary

Slow and sustained presence of selected drugs into the bone marrow is possible using this technology in order to achieve sustained presence of selected drugs over a period of days to weeks. Such agents could be delivered into the vascular space of long bones to provide chemotherapeutics to treat leukemia or growth factors to facilitate production. of progenitor blood cells. Such growth factors could include granulocyte colony stimulating factor (G-CSF), granulocyte/monocyte colony stimulating factor (GM-CSF), erythropoetin, thrombopoetin, interleukin-3.

19. Intra-Vascular Delivery

Slow and sustained presence of selected drugs into cardiovascular structures (arteries/veins) via lavage or catheter delivery is possible using this NHS/linker technology in order to achieve sustained presence of selected drugs over a period of days to weeks. Agents used for such purposes could include antithrombotics, antiproliferative agents (methotrexate, colchicine, angiopeptin, or heparin derivatives etc) to limit restenosis, protease inhibitors to limit vascular breakdown associated with aneurism and restenosis, radioactive therapy or DNA delivery to limit proliferation, angiostatic agents such as NHS derivatives of endostatin or angiostatin to limit vascular development or oligonucleotides, cDNA or naked DNA, and growth factors VEGF, FGF or to encourage vascular regeneration. Agents such as nitroso donors such as nitroprusside or other nitric oxide donors to restore normal vascular function in injured tissue would prove to be beneficial. Other agents such as oligosaccharides, cyclodextrans or mannose derivatives could be attached covalently to vasculature lumenal interface via NHS derivatization for the purpose of inhibiting leukocyte cell adhesion and rolling to limit vascular damage and inflammation in response to injury such as reperfusion, angioplasty or stent placement. Such agents would benefit from the derivatization with the reactive functionalities in order to facilitate placement and covalent adherence and enhance cellular loading and uptake to improve tissue retention and drug delivery.

Packaging

The therapeutic compound of the invention will be packaged in vials as a sterile liquid (preferably for room temperature storage), or as a lyophilisate in a vial for reconstitution. The solution (or diluent in the case of the lyophilisate) may contain a low concentration of some type of organic solvent in order to ensure solubility and stability. The liquid or post-reconstitution solution will contain as high a concentration as possible of the therapeutic compound. Each vial of therapeutic compound will contain an overage of therapeutic material required to treat each of the indications for the compound or to diagnose indications for the disease or condition.

Delivery Options

There are several delivery options for localized delivery of therapeutic agents.

a) Open surgical field lavage: There are a number of indications for local therapeutic compounds which would entail administration of the therapeutic compound as an adjunct to open surgery. In these cases, the therapeutic compound would either be lavaged in the surgical site (or a portion of that site) prior to closure, or the therapeutic compound would be incubated for a short time in a confined space (e.g., the interior of a section of an artery following an endarterectomy procedure or a portion of GI tract during resection) and the excess fluid subsequently evacuated.

b) Incubation of tissue grafts: Tissue grafts such as autologous and xenobiotic vein/artery and valve grafts as well as organ grafts can be pretreated with therapeutic compounds that have been modified to permit covalent bond formation by either incubating them in a therapeutic solution and/or perfusing them with such a solution.

c) Catheter delivery: A catheter is used to deliver the therapeutic compound either as part of an endoscopic procedure into the interior of an organ (e.g., bladder, GI tract, vagina/uterus) or adjunctive to a cardiovascular catheter procedure such as a balloon angioplasty. Standard catheters as well as newer drug delivery and iontophoretic catheters can be utilized.

d) Direct injection: For certain poorly vascularized spaces such as intra-articular joint spaces, a direct injection of a therapeutic compound may be able to bioconjugate to surface tissues and achieve a desirable duration of drug effect. Other applications could include intra medullary, intratumor, intravaginal, intrauterine, intra intestinal, intra eustachian tube, intrathecal, subcutaneous, intrarticular, intraperitoneal or intraocular injections as weel as via bronchoscope, via nasogastiric tube and via nophrostomy.

Patient Dosing

The compounds of this invention may be administered to a mammal, preferably a human. Because the delivery of a local therapeutic compound is targeted to achieve a local instead of a systemic effect, dosing as a function of patient weight or body surface area is not appropriate. For some therapeutic compounds, there is no significant risk of a local area safety or toxicity effect of the compound as a result of an overdosage. In such cases the therapeutic compound concentration formulation is optimized to deliver the highest possible density of conjugative bonding to the cells and proteins lining the targeted local area in order to retain therapeutic levels of localized drug activity at the site for as long a period of time as possible. However, some therapeutic compounds may have an optimal dose level (usually calculated based on conjugation density per surface area). In such cases, the density of conjugation per surface area is controlled as a function of a) surface site preparation, b) reaction time and conditions (e.g., temperature), c) therapeutic compound concentration of solution, and d) solution pH and buffers.

Administration Issues

The key factors in localized delivery of therapeutic agents are as follows: a) maintain the consistency of conjugation bonding density to the target tissues; b) minimize any local tissue irritation or other adverse effects of the therapeutic agent administration and c) minimize the amount of local therapeutic compound which goes systemic. To achieve these goals, it is important to prepare the site to eliminate as much of the surface “debris” (e.g., blood cells, loose proteins, etc.) as possible. This ensures that as much of the bioconjugated compound remains as close to the local site as possible, while ensuring uniformity of conjugation bonding density. Optimizing the compound solution, pH and buffers results in maximizing the level of conjugation bonding to the site surface. Removal of any residual liquid solution from an open operating field minimizes the amount of the therapeutic compound that may go systemic.

The invention can be more clearly illustrated by the following non-limiting examples.

### EXAMPLES

### Example 1

Synthesis of a NHS Derivative from a Carboxylic Acid in Absence of Other Sensitive Functionalities in the Molecule

To a solution of compound drug to be modified (1 mmol) containing a carboxylic acid in absence of other sensitive functionalities in the molecule and N-hydroxysuccinimide (1.1 mmol) in anhydrous CH₂Cl₂ (x mL) is added EDC (2.2 mmol). The solution is stirred at room temperature for 20 hours or until complete. The reaction is then washed with water, saturated sodium chloride, dried with anhydrous sodium sulfate, filtered and concentrated in vacuo. The residue is dissolved in minimum amount of solvent and purified by chromatography or recrystallized from the appropriate solvent system to give the NHS derivative.

### Example 2

Synthesis of an NHS Derivative from a Molecule Containing an Amino and/or a Thiol Functionality and a Carboxylic Acid

When a free amino or thiol group is present in the molecule, it is preferable to protect these functional groups prior to perform the addition of the NHS derivative. For instance, if the molecule contains a free amino group, a transformation of the amine into a Fmoc or preferably into a tBoc protected amine is necessary prior to perform the chemistry described in example 1. The amine functionality will not be deprotected after preparation of the NHS derivative. Therefore this method applies only to a compound whose amine group is not required to be freed to induce a pharmacological desired effect. If the amino group needs to be freed to retain the original biological properties of the molecule, then another type of chemistry described in example 3-6 has to be performed.

### Example 3

Synthesis of an NHS Derivative from a Molecule Containing an Amino or a Thiol Functionality and No Carboxylic Acid.

When the selected molecule contains no carboxylic acid, an array of bifunctional linker can be used to convert the molecule into a reactive NHS derivative. For instance, ethylene glycol-bis(succinimydylsuccinate) (EGS) and triethylamine dissolved in DMF and added to the free amino containing molecule (with a ratio of 10:1 in favor of EGS) will produce the mono NHS derivative. To produce an NHS derivative from a thiol derivatized molecule, one can use N-[γ-maleimidobutyryloxy]succinimide ester (GMBS) and triethylamine in DMF. The maleimido group will react with the free thiol and the NHS derivative will be purified from the reaction mixture by chromatography on silica or by HPLC.

### Example 4

Synthesis of a NHS Derivative from a Molecule Containing Multiple Chemical Functionalities

Each case will have to be analyzed and solved in a different manner. However, thanks to the large array of protecting groups and bifunctional linkers that are commercially available, this invention is applicable to any molecule with preferably one chemical step only to derivatize the molecule (as described in example 1 or 3) or two steps (as described in example 2 and involving prior protection of a sensitive group) or three steps (protection, activation and deprotection). Under exceptional circumstances only, would we require to use multiple steps (beyond three steps) synthesis to transform a molecule into an active NHS or maleimide derivative.

### Example 5

Synthesis of a Maleimide Derivative from a Molecule Containing a Free Amino Group and/or a Free Carboxylic Acid

To produce an maleimide derivative from a amino derivatized molecule, one can use N-[γ-maleimidobutyryloxy]succinimide ester (GMBS) and triethylamine in DMF. The succinimide ester group will react with the free amino and the maleimide derivative will be purified from the reaction mixture by crystallization or by chromatography on silica or by HPLC.

### Example 6

Synthesis of a Maleimide Derivative from a Molecule Containing Multiple Other Functionalities and No Free Carboxylic Acid

When the selected molecule contains no carboxylic acid, an array of bifunctional crosslinking reagentsl can be used to convert the molecule into a reactive NHS derivative. For instance maleimidopropionic acid (MPA) can be coupled to the free amine to produce a maleimide derivative through reaction of the free amine with the carboxylic group of MPA using HBTU/HOBt/DIEA activation in DMF.

Many other commercially available heterobifunctional crosslinking reagents can alternatively be used when needed.

### Example 7

Preparation of Rhodamine NHS Ester

Rhodamine Green™-X, succinimidyl ester, hydrochloride mixed isomers is commercially available from Molecular Probes (Eugene Oreg.) as illustrated below:

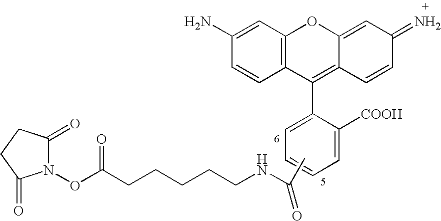

### Example 8

In Vivo Addition of NHS-Rhodamine

New Zealand rabbits (2 Kg), male or female, were intramuscularly anesthetized with Xylazine (20 mg/kg), Ketamine (50 mg/kg) and Acepromazine (0.75 mg/kg) prior to surgical exposure of left carotid artery. Both carotid arteries were isolated and blood flows were measured. A catheter (22G) was inserted in the arterial segment and rinsed with 0.9% sodium chloride via catheter until there was no more visible evidence of blood in the segment.

A 1-cm incubation chamber was created by ligatures in the segment area. The incubation chamber was flushed three times with 1 mL of 0.9% sodium chloride. A solution of 100 μl of 500 μM NHS-Rhodamine was prepared and incubated in the incubation chamber for 3 minutes. The excess of rhodamine was withdrawn with a 1 mL syringue. The incubation chamber was washed once again with 3 times 100 mL of 0.9% sodium chloride. The incubation chamber was then removed from the rabbit, cut in three pieces and dipped in 10% formalin for further evaluation. The NHS-Rhodamine treated arteries exhibited dramatic levels of fluorescence whereas those arteries treated solely with Rhodamine exhibited little fluorescence over background. These results demonstrate that Rhodamine was covalently bonded to a local delivery site.

### Example 9

Preparation of [³H]-NHS-Propionate

[³H]-NHS-propionate is available from Amersham Canada Ltd. (Oakville, Ontario, Canada) and can be prepared from the tritiated propionic acid through known to the art condensation of N-hydrosuccinimide in presence of EDC in DMF or methylene chloride.

### Example 10

In Vivo Pharmacokinetics Studies of [³H]-NHS-Propionate

New Zealand rabbits (2 kg), male or female, were intramuscularly anesthetized with Xylazine (20 mg/kg), Ketamine (50 mg/kg) et Acepromazine (0.75 mg/kg) prior to surgical exposure of left carotid artery. Segments of 10 mm of carotids, were transiently isolated by temporary ligatures and rinsed with C.9% sodium chloride via a cannula until there was no more visible evidence of blood components.

A catheter (18G) was inserted in the arterial segment and served to introduce the angioplasty balloon (2.5 mm of diameter, over the wire/Boston Scientific Inc.). A vascular damage (angioplasty) was performed on the isolated segment in order to eliminate the layer of endothelial cells. The angioplasty balloon was serially inflated at different atmospheres (4, 6, 8 and 10) during 1 minute, with 45 seconds of delay between inflations. At 4 atmospheres a balloon traction was performed 5 times and 1000 U/kg of heparin were infused in the blood circulation.

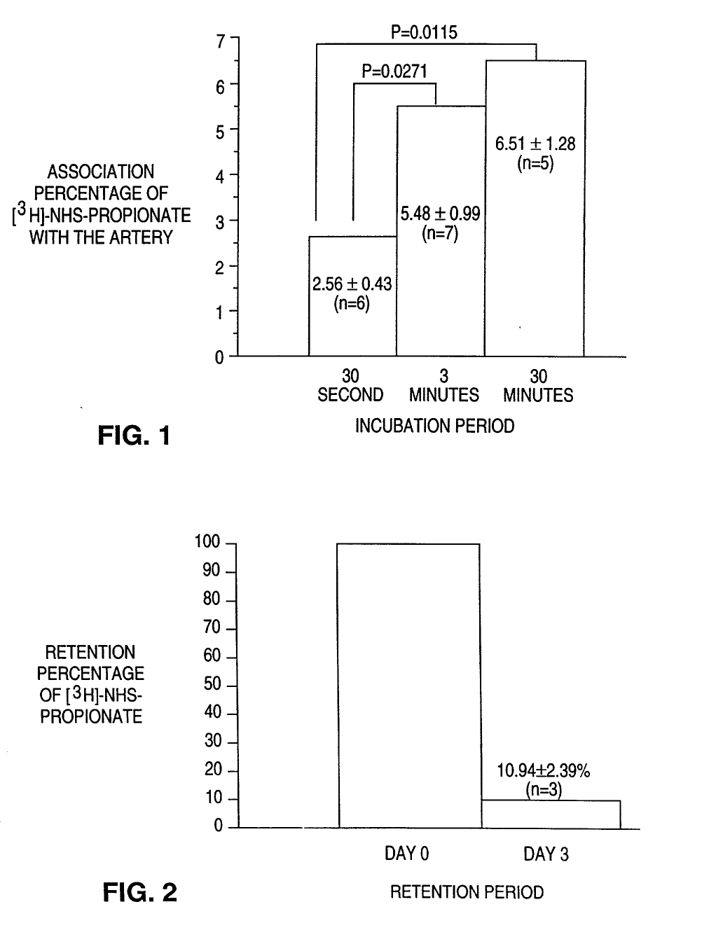

The angioplasty balloon was then retrieved from the artery and the catheter was reintroduced. The arterial segment was rinsed 3 times with saline, and 100 μM of [³H]-NHS-propionate was incubated within the isolated segment of the artery for either 30 seconds, 3 minutes or 30 minutes. At the end, the excess of incubation liquid was withdrawn from the artery, and the segment was rinsed 5 times with saline. The treated artery was immediately harvested, and incorporation of [³H]-labeled compounds within the artery was evaluated by scintillation counting (see FIG. 1). After 30 seconds of incubation, we recorded an association efficiency of 2.55%. At 3 min and 30 min, we recorded an association efficiency of 5.5 and 6.5%, respectively. We decided that a 3 min incubation time was sufficient to treat the artery in an efficient way.

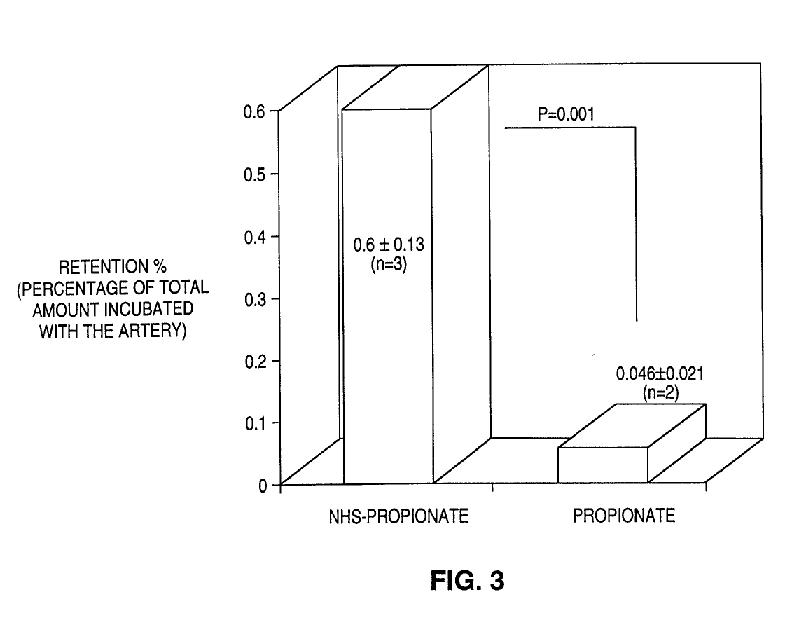

When evaluating the retention levels, 100 μM of [³H]-NHS-propionate or [³H]-propionate were incubated with the artery for a period of 3 minutes, after which the segment has been rinsed 5 times with saline. The catheter was then removed and the arteriotomy site was closed with microsutures, thus reestablishing the blood flow within the carotid. Finally, the neck wound was closed with sutures, and animals are allowed to recuperate. Three days following the treatment, the animals are sacrificed with an overdose of sodium pentobarbital, the carotid segments are removed and examined for compound's presence by scintillation counting. As shown on FIG. 2, 10.94% retention of [3H]-NHS-propionate was monitored after three days following a 3 minute incubation period based on residual radioactivity in the artery. FIG. 3 shows the difference in retention efficiency between covalently and non covalently bound propionate after a 3 minutes incubation period. An outstanding 12 fold enhancement in retention was recorded (0.6% of total amount incubated against 0.046% for the non covalently bound) in favor of the NHS-propionate. This indicates that the tissue association of a compound is dramatically enhanced by the covalent attachment in vivo. Subsequent restitution of blood flow demonstrated retention [³H]-NHS-propionate of approximately 10% of the material 72 hours after injury (FIG. 2). This represents excessive tissue retention using the embodied technology of agents markedly beyond that seen with all local drug delivery technologies as exemplified in the literature for standard non covalent agents (Circulation 1994 89 (4) 1518-1524).

### Example 11

Synthesis of [³²P] NHS Derivative

To a solution of protected R and R′ (both R and R′ can be

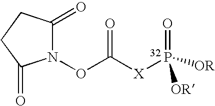

alkyl, phenyl or alkoxy groups, and X is either O or S, alkoxy, alkyl and any other functionality stable under these conditions) phosphodiester (0.1 mmol) and N-hydroxysuccinimide (0.2 mmol) is added diisopropylethylamine 0.11 mmol), followed by addition of HBTU (0.22 mmol). The reaction mixture is stirred at room temperature for 36 hours. DMF is removed by vacuum distillation and the residue is dissolved in MeOH (10 mL). The MeOH solution is filtered to remove the insolubles, the filtrate is concentrated in vacuo, and the residue is dissolved in a minimum amount of MeOH. Water is then added to induce precipitation and the precipitate is dried on vacuum to give the desired compound

The yield of the reaction can usually be improved by using EDC as the coupling reagent, as exemplified below. To a solution of R and R′ phosphodiester (0.054 mmol) and N-hydroxysuccinimide (0.115 mmol) in anhydrous DMF (3 mL), is added EDC (31 mg, 0.162 mmol). The solution is stirred at room temperature for 24 hours. DMF is removed by vacuum distillation and the residue is further dried on high vacuum. The residue is then dissolved in a minimum amount of MeOH (0.12 mL) and H₂O (3.2 mL) is added to induce precipitation. The precipitates are washed with H₂O (3×0.8 mL) and dried on vacuum to give a solid product.

Any protected phosphonate derivatives may undergo similar transformation.

### Example 12

New Zealand rabbits (2 kg), male or female, were anesthetized with xylazine (20 mg/kg), ketamine (50 mg/kg) and acepromazine (0.75 mg/kg) intramuscularly prior to surgical exposure of left carotid artery. Carotid arteries were surgically dissected and segments of approximately 10 mm length were isolated. The vessels were cannulated and rinsed with 0.9% sodium chloride until there was no more visible evidence of blood components.

A catheter (18G) was inserted in the arterial segment and served to introduce the angioplasty balloon (2.5 mm of diameter, over the wire/Boston Scientific Inc.). Vascular damage (angioplasty) was performed on the isolated segment in order to eliminate the layer of endothelial cells. The angioplasty balloon was serially inflated at different atmospheres (4, 6, 8 and 10) for 1 minute, with 45 seconds of delay between inflations. At 4 atmospheres a balloon traction was performed 5 times and 1000 U/kg of heparin were infused in the blood circulation.

The angioplasty balloon was then retrieved from the artery and the catheter was reintroduced. The arterial segment was rinsed 3 times with saline, and 100 μM of [³²P] NHS-[linker] was incubated within the isolated segment of the artery for 3 minutes. At the end, the excess of incubation liquid was withdrawn from the artery, and the segment was rinsed 5 times with saline. The vessel was sutured closed, blood flow restored and surgical wounds repaired. Animals were returned to the vivarium for periods up to four weeks. Tissue retention of [³²P]-NHS-[linker] was evaluating using whole animal radiography at selected periods of time after injury. Tissue response to this therapy can be evaluated using standard histomophometric analysis quantifying if the extent of tissue proliferation and neoitimal formation in the treated versus control animals to determine if this form of brachotherapy can limit the response to vascular injury and hyperproliferative overgrowth classical observed under these conditions.

### Example 13

Synthesis of [131]-NHS Derivative

To a solution of protected amino protected [¹³¹I]-iodotyrosine (0.1 mmol) and N-hydroxysuccinimide (0.2 mmol) is added diisopropylethylamine (0.11 mmol), followed by addition of HBTU (0.22 mmol). The reaction mixture is stirred at room temperature for 12 hours. DMF is removed by vacuum distillation and the residue is dissolved in MeOH (10 mL). The MeOH solution is filtered to remove the insolubles, the filtrate is concentrated in vacuo, and the residue is dissolved in a minimum amount of MeOH. Water is then added to induce precipitation and the precipitate is dried on vacuum to give the desired compound

The yield of the reaction can usually be improved by using EDC as the coupling reagent, as exemplified below. To a solution of [¹³¹I]-iodotyrosine (0.054 mmol) and N-hydroxysuccinimide (0.115 mmol) in anhydrous DMF (3 mL), is added EDC (31 mg, 0.162 mmol). The solution is stirred at room temperature for 24 hours. DMF is removed by vacuum distillation and the residue is further dried on high vacuum. The residue is then dissolved in a minimum amount of MeOH (0.12 mL) and water (3.2 mL) is added to induce precipitation. The precipitates are washed with H₂O (3×0.8 mL) and dried on vacuum to give a solid product.

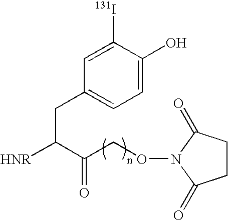

### Example 14

In Vivo Pharmacology of ¹³¹I Derivative

New Zealand rabbits (2 Kg), male or female, were anesthetized with xylazine (20 mg/kg), ketamine (50 mg/kg) and acepromazine (0.75 mg/kg) intramuscularly prior to surgical exposure of left carotid artery. Carotid arteries were surgically dissected and segments of approximately 10 mm length were isolated. The vessels were cannulated and rinsed with 0.9% sodium chloride until there was no more visible evidence of blood components.

A catheter (18G) was inserted in the arterial segment and served to introduce the angioplasty balloon (2.5 mm of diameter, over the wire/Boston Scientific Inc.). Vascular damage (angioplasty) was performed on the isolated segment in order to eliminate the layer of endothelial cells. The angioplasty balloon was serially inflated at different atmospheres (4, 6, 8 and 10) for 1 minute, with 45 seconds of delay between inflations. At 4 atmospheres a balloon traction was performed 5 times and 1000 U/kg of heparin were infused in the blood circulation.

The angioplasty balloon was then retrieved from the artery and the catheter was reintroduced. The arterial segment was rinsed 3 times with saline, and 100 μM of [¹³¹I]-NHS-[linker] was incubated within the isolated segment of the artery for 3 minutes. At the end, the excess of incubation liquid was withdrawn from the artery, and the segment was rinsed 5 times with saline. The vessel was sutured closed, blood flow restored and surgical wounds repaired. Animals were returned to the vivarium for periods up to four weeks. Tissue retention of [¹³¹I]-NHS-[linker] was evaluated using whole animal radiography at selected periods of time after injury. Tissue response to this therapy can be evaluated using standard histomophometric analysis quantifying if the extent of tissue proliferation and neoitimal formation in the treated versus control animals to determine if this form of brachotherapy can limit the response to vascular injury and hyperproliferative overgrowth classical observed under these conditions.

### Example 15

General

Products from the following examples were purified by preparative reversed phase HPLC using a Varian (Rainin) preparative binary HPLC system: gradient elution of 5-60% B (0.045% TFA in H₂O (A) and 0.045% TFA in CH₃CN (B)) at 9.5 mL/min using a Dynamax C₁₈, 60 Å, 8 μm, 21 mm×25 cm column equipped with a Dynamax C₁₈, 60 Å, 8 μm guard module and a UV detector (Varian Dynamax UVD II) detecting at λ214 and 254 nm. Analytical HPLC were performed using a Varian (Rainin) binary HPLC system: gradient elution of 5-60% B (0.045% TFA in H₂O (A) and 0.045% TFA in CH₃CN (B)) at 0.5 mL/min using a Dynamax C₁₈, 60 Å, 8 μm, 4.6 mm×25 cm column equipped with a Dynamax C₁₈, 60 Å, 8 μm guard module and an UV detector (Varian Dynamax UVD II) detecting at λ214 and 254 nm. Mass spectrometry was performed on a PE Sciex API III electro-spray Biomolecular Mass Analyzer.

### Example 16

Synthesis of Ac-RIARGDFPDDRK-NH₂.4 TFA Syntheses of Ac-RIARGDFPDDRK-NH₂ peptide was performed on an ABI 433A Peptide Synthesizer using 510 mg of 0.49 mmol/g of Fmoc protected Rink Amide MBHA resin (NovaBiochem), 4 eq. of Fmoc protected amino acids, 4 eq. of a 0.45 M O-benzotriazol-1-yl-N,N,N′,N′-tetramethyl-uronium hexafluorophosphate (HBTU) and 1-hydroxybenzotriazole (HOBt) in N,N-dimethylformamide solution as activation with 4 eq. of 2 M N,N,-diisopropylethylamine (DIEA) in 1-methyl-2-pyrrolidinone (NMP), and piperidine deprotection of Fmoc groups. Upon completion of the sequence, the resin was dried to afford 990 mg of a tan resin (91%).

The peptide was removed from the resin by shaking 609 mg of Ac-RIARGDFPDDRK-MBHA-Resin with two-5 mL portions of a cleavage cocktail (comprised of: 10 mL of trifluoroacetic acid (TFA); 0.75 g of phenol; 0.25 g of thioanisole; 0.5 mL of ethanedithiol (EDT); and 0.5 mL of water) for 2 h each. The filtrates were each collected and combined and combined with the filtrates from washing the resin with 5 mL of TFA and 5 mL of CH₂Cl₂. The filtrates were then concentrated to approximately 10 mL and the product was precipitated out by the addition of 40 mL of dry-ice cold Et₂O. The resulting precipitate was collected by centrifugation and re-suspended in 40 mL of dry-ice cold Et₂O, centrifuged and process repeated to afford 163 mg of the crude peptide as a white solid (0.084 mmol, 60%). Analytical HPLC showed purity to be approximately 72%.

### Example 17

Synthesis of Ac-RIARGDFPDDRK(EGS)-NH₂.3 TFA

In a 15-mL centrifuge tube, 46.2 mg of ethylene glycol-bis(succinimidylsuccinate) (EGS) (0.071 mmol) and 8.50 μL of triethylamine (0.061 mmol) was dissolved in 500 μL of DMF. To this vortexing solution was added dropwise over 30 sec a solution of 12.2 mg of Ac-RIARGDFPDDRK-NH₂.4 TFA (0.006 mmol) (Example 2) in 100 μL of DMF and following addition the reaction was allowed to stand at RT for 1.5 h. To this was added 9.88 μL of TFA (0.122 mmol), vortexed and product precipitated out by the addition of 15-mL of dry-ice cold Et₂O. The precipitate was collected by centrifugation and solid taken up in 1 mL of 0.045% TFA in CH3CN and 1 mL 0.045% TFA in water and deposited on prep HPLC and desired fractions collected and lyophilized to afford 6.80 mg of product as a white solid (0.003 mmol, 50%). Analytical HPLC indicated product to be>70% pure with Rₜ=38.17 min (product) and 37.21 min (hydrolysis product) ESI-MS m/z for C₇₇H₁₁₉N₂₄O₂₈ (MH⁺), calcd. 1827.9, found MH²+914.8. Hydrolysis product ESI-MS m/z for C₇₃H₁₁₆N₂₃O₂₆ (MH⁺), calcd. 1731.9, found MH²⁺ 866.2.

### Example 18

Cell Culture

Human umbilical vein endothelial cells (HUVEC) from ATCC are grown to confluence in Medium 199 containing 2.2 mg/mL of sodium bicarbonate supplemented with 20% heat-inactivated FBS, ECGS 150 μg/mL, penicillin 100 Ui/mL, streptomycin 100 μg/mL. Cells are grown in 75 cm² flask at 37° C. under 5% CO₂ and the medium is replaced on the first day of seeding and every two days there after.

The cells are used between second and fourth passages. Selected RGD containing agents are evaluated for their ability to inhibit the growth and proliferation of these cells under normal conditions as well as in confluent cultures that are injured mechanically by scraping a lesion into the confluence and measuring the rate of wound healing or trough repopulation.

### Example 19

In Vitro Cell Adhesion Assay

Standard 48-well cell culture plates (Costar) are coated with 100 μL fibronectin (5 and 10 μg/mL), vitronectin (5-10 μg/mL) or HSA 1 in PBS overnight and air dried. Endothelial and somatic cells are harvested by treating with trypsin (0.25%, w/v)/EDTA (1 mM) (5 mL/25 cm² of surface area), washed twice in PBS and suspended at 5×10⁵ cells/mL in PBS containing 10 μg/mL of fluorescein isothiocyanate (FITC) for 30 minutes at 37° C.

Labeled cells are washed and re suspended at 5×10⁵ cells/mL in incubation medium Ml 99, then incubated with different concentrations of the HSA-RGD peptide for 30 minutes at 37° C.

Control and pretreated cells are applied into ECM-coated plates at a density of 5×10⁴ cells/well and incubate for 90 minutes at 37° C. allowing adhesion to occur. After washing twice with PBS, non adherent cells are removed by aspiration, and plates are subjected to quantification of fluorescence density using the SpectroMax. Following detachment by trypsin, the adherent cells are also counted by hemacytometer and the percentage of cells adhering to ECM is calculated. The direct effect of HSA-RGD on cell adhesion in this assay is a tool to define effects not only in wound healing but in inhibiting platelet and leukocyte adhesion and diapedesis following adhesion.

### Example 20

In Vitro Cell Migration Assay

Cells are harvested by treating with trypsin/EDTA (5 mL/25 cm² of surface area), washed twice in PBS and suspended at 5×10⁵ cells/mL in PBS containing different concentrations of the HSA-RGD peptide. The isolated cells are Incubate for 30 minutes at 37° C. and then added to the upper chamber of the Transwell (Costar 8.0 μm) chamber that had been precoated with a type I collagen as a chemotractant. After 6-18 hours of incubation at 37° C., 5% CO₂ the membrane insert was recovered and cells remove with Q-tip cotton swab and placed into 1% crystal violet in 20% methanol 80% water. The cells were stained for 20 minutes and extracted with 10% acetic acid and absorbance measured at 600 nm. The number of migrated cells are calculated from a standard curve.

### Example 21

Matrigel-Induced Capillary Formation

Endothelial cells spontaneously form capillary tube when seeded on natural ECM. Basement membrane matrix (Sigma) is diluted at 4 mg/mL with cold PBS and added to 24-well plates (Costar) in a total volume of 200 μL in each well. Plates are left at 37° C. for 30 minutes to form a gel layer into which HUVEC cells 2×10⁵ in a medium with 20% FBS supplemented with concentrations of peptides are applied to each well and incubated at 37° C. for 24 hours with 5% CO₂. After incubation, cells are washed, fixed in 2% glutaldehyde for 10 minutes and subjected to inverted contrast-phase microscopy. The effect of the RGD peptides on tube forming sprouts are visually measured to determine the effects on capillary formation.

### Example 22

Chicken CAM (Chorioallantoic Membrane) Assay

The CAM in the chick embryo is a classical in ovo assay to quantitate drug effect on vascular angiogenesis. Ten days old chicken embryos are incubated at 37° C. with 60% humidity. A 1 cm² window is made to access the underlying CAM. Angiogenesis is induced by injecting 200 μL of TNF α, 5 ng/mL on the CAM. After 24 hours, test RGD peptides are applied in a volume of 50 μL. The window is covered with sterile cellophane tape and the embryos are incubated for a further 48 hours at 37° C. with 60% humidity. After incubation, CAM tissue is resected and angiogenesis is visualized on microscope. The inhibition of vascular formation is semi quantitively scored to assess the effects of the RGD peptides in inhibiting angiogenesis.

### Example 23

Corneal Neovascularization Bioassay

Rats are anesthetized and small pocket is made on the cornea to inserted material to induce blood vessel formation. Pellets of a slow releasing polymer (Hydron) containing angiogenic factor (VEGF) and different concentrations of RGD peptides are implanted into the pocket. (See preparation mode of mixture in Hydron in D'Amato technique. Angiogenesis 1996). After 3 to 5 days, the animals will be killed and corneal vessel is photographed. The density of blood vessel growth is scored and activity of test RGD peptides defined as reflected by the extent of inhibiting blood vessel formation.

### Example 24

In Vivo Wound Healing Activity

Nude mice are anesthetized with sodium pentobarbital (25 mg/kg ip) and skin lesions produced with 0.5 mm silver nitrate cautery device. The lesion produced heals in 3-5 days. The rate of cellular infiltrate and the limitation of scarring is assessed in animals treated with RGD peptides to facilitate wound healing. In advanced animal models RGD peptides for surgical wound healing will be evaluated in models of gastrointestinal surgery. Under surgical anesthesia rats with undergo a laparotomy and a complete transection of the duodenum. AT the time of surgery the cut and adjacent ends are treated with RGD peptides and resutured. The time to healing is assessed in comparison to controls based upon the rate of histopathological healing as well as restitutioning GI function as assessed by charcoal transit times evaluated in these animals 24-72 hours after surgical injury. The ability of the RGD peptides to heal the cut ends of the GI tract will be characterized over a several week period of time to assess the enhancement of the healing process.

### Example 25

In Vivo Angiogenesic and Anti-Metastatic Activity

The anti-metastatic activity of such RGD peptides is evaluated in nude mice inoculated with different human cancer cell lines. In this assays the tumor are established to a defined mass greater than 2 cm using defined growth and mass curves. Animals are injected with the NHS RGD peptides directly into the established human tumor and the effects of the local application of the RGD peptide on the progression of tumor size and mass is determined in comparison to vehicle treated animals. The anti-proliferative activity and effects on angiogenesis is determined by direct quantification of capillary formation, cell number, tumor density and blood flow defined into the tumor after treatment.

### Example 26

In Vivo Anti Restenosis Activity

New Zealand rabbits (2 Kg), male or female, were intramuscularly anesthetized with Xylazine (20 mg/kg), Ketamine (50 mg/kg) et Acepromazine (0.75 mg/kg) prior to surgical exposure of left carotid artery. Segments of 10 mm of carotids, were transiently isolated by temporary ligatures and rinsed with C.9% sodium chloride via a cannula until there was no more visible evidence of blood components. The carotid artery is injured by standard balloon angioplasty and RGD applied at the time of injury. The surgical incisions are repaired and the animals returned to the vivarium for periods of time up to one month after injury.

At the time of terminal sacrifice the injured vessel is isolated and inspected with perfusion fixation and harvesting. The response to injury is assessed histomorphologically using computer imaging of the cross sectional areas and calculating the intimal to medial ratios as well as evaluating using BrDU the extent of cellular proliferation in this in vivo assay. The effects of local RGD peptides on preventing the hyperproliferative response in this model reveals drug interactions in stabilizing the injured vessel.

### Example 27

Ex Vivo Vascular Grafting

New Zealand rabbits (2 kg), male or female, were intramuscularly anesthetized with Xylazine (20 mg/kg), Ketamine (50 mg/kg) et Acepromazine (0.75 mg/kg) prior to surgical exposure of left jugular vein. Segments of 20 mm of carotids, were transiently isolated by temporary ligatures and rinsed with 0.9% sodium chloride via a cannula until there was no more visible evidence of blood components. The vessels are removed and surgical transplanted into the descending aorta. Twenty four hours after surgery the animals are reanesthetized and blood flow measured using Transonic flow probes to determine if thrombosis has occurred. The application of NHS RGD to the vascular graft can act as a procoagulant or an anticoagulant. The local application of the RGD will be measured in different uses to prevent bleeding at the suture sites as well as to define the effects on platelet deposition and arterialization at various time points in this model.

The invention now being fully described, it will be apparent to one of ordinary skill in the art that many changes and modifications can be made thereto without departing from the spirit or scope of the appended claims.

We claim:

1. A local delivery agent comprising a compound of the formula:
X—Y-Z
wherein X is selected from the group consisting of wound healing agents, antibiotics, anti-inflammatories, antioxidants, antiproliferatives, immunosuppressants, anti-infective and anti-cancer agents;
Y is a linking group consisting of 0-30 atoms; and
Z is a chemically reactive entity capable of reaction with a reactive functionality on fixed blood components to form covalent bonds therewith.

2. The composition of claim 1 wherein said fixed blood component is a protein.

3. The composition of claim 1 wherein said reactive functionality is selected from the group consisting of an amino group, a carboxyl group or a thiol group.

4. The composition of claim 1 wherein Z is selected from the group consisting of N-hydroxysuccinimide, N-hydroxy sulfosuccinimide, maleimide-benzoyl-succinimide, gamma-maleimido-butyryloxy succinimide ester, maleimidopropionic acid, isocyanate, thiolester, thionocarboxylic acid ester, imino ester, carbodiimide anhydride and carbonate ester.

5. The composition of claim 5 wherein Z is N-hydroxysuccinimide.

6. The composition of claim 1 wherein X is a peptide.

7. The composition of claim 1 wherein X is an organic molecule.

8. The composition of claim 1 wherein X contains a radioactive isotope.

9. A local delivery agent comprising a compound of the formula:
X—Y-Z
wherein X is selected from the group consisting of wound healing agents, anti-inflammatories, antiproliferatives, and chemotherapeutic agents;
Y is a linking group consisting of 0-30 atoms; and
Z is a chemically reactive entity capable of reaction with a reactive functionality on fixed blood components to form covalent bonds therewith.

10. The composition of claim 9 wherein said fixed blood component is a protein.

11. The composition of claim 9 wherein said reactive functionality is selected from the group consisting of an amino group, a carboxyl group or a thiol group.

12. The composition of claim 9 wherein Z is selected from the group consisting of N-hydroxysuccinimide, N-hydroxy sulfosuccinimide, maleimide-benzoyl-succinimide, gamma-maleimido-butyryloxy succinimide ester, maleimidopropionic acid, isocyanate, thiolester, thionocarboxylic acid ester, imino ester, carbodiimide anhydride and carbonate ester.

13. The composition of claim 9 wherein Z is N-hydroxysuccinimide.

14. The composition of claim 9 wherein X is a peptide.

15. The composition of claim 9 wherein X is an organic molecule.

16. The composition of claim 9 wherein X is a radiolabeled element.

17. A wound healing agent comprising a compound of the formula:
X—Y-Z
wherein X is a therapeutic agent that has wound healing properties;
Y is a linking group consisting of 0-30 atoms; and
Z is a chemically reactive entity capable of reaction with a reactive functionality on fixed blood components to form covalent bonds therewith.

18. The composition of claim 17 wherein said fixed blood component is a protein.

19. The composition of claim 17 wherein said reactive functionality is selected from the group consisting of an amino group, a carboxyl group or a thiol group.

20. The composition of claim 17 wherein Z is selected from the group consisting of N-hydroxysuccinimide, N-hydroxy sulfosuccinimide, maleimide-benzoyl-succinimide, gamma-maleimido-butyryloxy succinimide ester, maleimidopropionic acid, isocyanate, thiolester, thionocarboxylic acid ester, imino ester, carbodiimide anhydride and carbonate ester.

21. The composition of claim 17 wherein Z is N-hydroxysuccinimide.

22. A wound healing agent comprising a compound of the formula:
X—Y-Z
wherein X is an RGD containing peptide have wound healing properties;
Y is a linking group consisting of 0-30 atoms; and
Z is a chemically reactive entity capable of reaction with a reactive functionality on fixed blood components to form covalent bonds therewith.

23. The composition of claim 22 wherein said fixed blood component is a protein.

24. The composition of claim 22 wherein said reactive functionality is selected from the group consisting of an amino group, a carboxyl group or a thiol group.

25. The composition of claim 22 wherein Z is selected from the group consisting of N-hydroxysuccinimide, N-hydroxy sulfosuccinimide, maleimide-benzoyl-succinimide, gamma-maleimido-butyryloxy succinimide ester, maleimidopropionic acid, N-hydroxysuccinimide, isocyanate, thiolester, thionocarboxylic acid ester, imino ester, carbodiimide anhydride and carbonate ester.

26. The composition of claim 22 wherein Z is N-hydroxysuccinimide.

27. The composition of claim 22 wherein the RGD containing peptide is:
Ac-RIARGDFPDDRK(EGS)-NH₂
where EGS is ethylene glycol-bis(succinimidylsuccinate)

28. A local delivery agent comprising a compound of the formula:
X—Y-Z
wherein X is an anti-restenosis, antiproliferative or an antiangiogenic agent wherein said agent is radioactive, wherein
Y is a linking group consisting of 0-30 atoms; and
Z is a chemically reactive entity capable of reaction with a reactive functionality on a fixed blood component to form covalent bonds therewith.

29. The composition of claim 28 wherein said fixed blood component is a protein.

30. The composition of claim 28 wherein said reactive functionality is selected from the group consisting of an amino group, a carboxyl group or a thiol group.

31. The composition of claim 28 wherein Z is selected from the group consisting of N-hydroxysuccinimide, N-hydroxy sulfosuccinimide, maleimide-benzoyl-succinimide, gamma-maleimido-butyryloxy succinimide ester, maleimidopropionic acid, isocyanate, thiolester, thionocarboxylic acid ester, imino ester, carbodiimide anhydride and carbonate ester.

32. The composition of claim 28 wherein Z is N-hydroxysuccinimide.

33. A local delivery agent comprising a compound of the formula:
X—Y-Z
wherein X is an anti-restenosis, an antiproliferative or an antiangiogenic agent wherein said agent contains an RGD peptide
Y is a linking group consisting of 0-30 atoms; and
Z is a chemically reactive entity capable of reaction with a reactive functionality on fixed blood components to form covalent bonds therewith.

34. The composition of claim 33 wherein said fixed blood component is a protein.

35. The composition of claim 33 wherein said reactive functionality is selected from the group consisting of an amino group, a carboxyl group or a thiol group.

36. The composition of claim 33 wherein Z is selected from the group consisting of N-hydroxysuccinimide, N-hydroxy sulfosuccinimide, maleimide-benzoyl-succinimide, gamma-maleimido-butyryloxy succinimide ester, maleimidopropionic acid, isocyanate, thiolester, thionocarboxylic acid ester, imino ester, carbodiimide anhydride and carbonate ester.

37. The composition of claim 33 wherein Z is N-hydroxysuccinimide.

38. The composition of claim 33 wherein the RGD peptide is:
Ac-RIARGDFPDDRK(EGS)-NH₂
wherein EGS is ethylene glycol-bis(succinimidylsuccinate) and Ac is an acetylated terminal amino acid.

39. A local delivery agent comprising a compound of the formula:
X—Y-Z
wherein X is an anti-restenosis, an antiproliferative or an antiangiogenic agent wherein said agent includes a radioactive isotope, wherein
Y is a linking group consisting of 0-30 atoms; and
Z is a chemically reactive entity capable of reaction with a reactive functionality on a fixed blood component to form covalent bonds therewith.

40. The composition of claim 39 wherein said fixed blood component is a protein.

41. The composition of claim 39 wherein said reactive functionality is selected from the group consisting of an amino group, a carboxyl group or a thiol group.

42. The composition of claim 39 wherein Z is selected from the group consisting of N-hydroxysuccinimide, N-hydroxy sulfosuccinimide, maleimide-benzoyl-succinimide, gamma-maleimido-butyryloxy succinimide ester, maleimidopropionic acid, isocyanate, thiolester, thionocarboxylic acid ester, imino ester, carbodiimide anhydride and carbonate ester.

43. The composition of claim 39 wherein Z is N-hydroxysuccinimide.

44. The composition of claim 39 wherein said radioactive isotope is a beta ray or a gamma ray emitter.

45. A method of increasing the retention time of a therapeutic agent locally administered to a site, comprising:
delivering to a localized site in a mammal a compound according to claim 3 of the formula:
X—Y-Z
wherein:
X is a therapeutic agent selected from the group consisting of wound healing agents, antibiotics, anti-inflammatories, antioxidants and chemotherapeutic agents;
Y is a linking group of 0-30 atoms; and
Z is a chemically reactive group capable of reaction with a reactive functionality of said site to form one or more covalent bonds therewith.

46. The method of claim 32 wherein said device is selected from the group consisting of syringes, catheters, trocars and endoscopes.

47. The method of claim 32 wherein said formulation is delivered intravascularly.

48. The method of claim 33 wherein said formulation is delivered topically.

49. The method of claim 33 wherein said formulation is delivered intraarterially.

50. The method of claim 45 wherein said mammal is a human.

51. A method of promoting wound healing at a wound site, comprising:
applying a compound of the formula X—Y-Z wherein X is a wound healing agent, Y is a linking group between 0-30 atoms and Z is a chemically reactive entity capable of reaction with a reactive functionality on fixed blood components to form covalent bonds therewith, wherein said compound is applied at or near said site to permit covalent bond formation of said compound to a reactive functionality near said site.

52. A method of treating a tumor, comprising:
applying a compound of the formula X—Y-Z wherein X is an anti-cancer agent, Y is a linking group between 0-30 atoms and Z is a chemically reactive entity capable of reaction with a reactive functionality on fixed blood components to form covalent bonds therewith, wherein said compound is applied at or near said tumor to permit covalent bond formation of said compound to a reactive functionality at or near said tumor.
